(19) United States
(12) Patent Application Publication
(10) Pub. No.: US 2019/0282349 A1
(43) Pub. Date: Sep. 19, 2019

(54) BIASING SEX SELECTION

(71) Applicant: BIASSEX PTY LTD, West Perth, Western Australia (AU)

(72) Inventor: Josef PFISTERSHAMMER, Claremont (AU)

(73) Assignee: BIASSEX PTY LTD, West Perth, Western Australia (AU)

(21) Appl. No.: 16/343,688

(22) PCT Filed: Sep. 20, 2017
(86) PCT No.: PCT/AU2017/000201

§ 371 Date: Apr. 19, 2019

(30) Foreign Application Priority Data
Sep. 21, 2016 (AU) 2016903813

(51) Int. Cl. A61D 19/00; C12N 5/071; C07K 16/18; A61K 47/26; A61K 47/50

(52) U.S. Cl. CPC A61D 19/00; C12N 5/0612; A61K 47/50; A61K 47/26; C07K 16/18

(57) ABSTRACT

A method for biasing sex selection, the method comprising the introduction into a uterus of a subject a volume of micro-particle conjugates (10), wherein the micro-particle conjugates are proportioned so as to approximate the size and shape of spermatozoa and thereby be carried by peristaltic waves through the uterus and fallopian tubes to the infundibulum, at which point any spermatozoa present or arriving thereafter undergo capacitation and expose antigens that may be bound by anti-male antibodies (14) provided in the micro-particle conjugates (10), the binding of the exposed antigens on the spermatozoa by the anti-male antibodies resulting in their inability to penetrate the Zona Pellucida of an egg and effect fertilisation.

### CROSS-REFERENCE TO RELATED APPLICATIONS

This application is a 371 U.S. National Stage of International Application No. PCT/AU2017/000201, filed on Sep. 20, 2017, which claims priority to Australian Application No. 2016903813, filed on Sep. 21, 2016. The entire disclosures of the above applications are incorporated herein by reference.

### FIELD OF THE INVENTION

The present invention relates to a method for biasing sex selection and apparatus for achieving same.

More particularly, the method and apparatus for biasing sex selection of the present invention are intended to have application in both natural mating and artificial insemination. In both circumstances it is understood that the method and apparatus may be applied to unsexed semen.

The present invention further provides a micro-particle conjugate for use in the method of the present invention. More particularly, the micro-particle conjugate of the present invention is intended to facilitate the passage of anti-male antibodies to a point in the female uterine tract at which sperm are capacitated, thereby exposing antigens thereon and in turn allowing the anti-male antibodies to bind to same.

### BACKGROUND

In livestock industries it is common to utilise artificial insemination for the genetic improvement, particularly in cattle. For example, it is possible to utilise the semen of a few highly selected males to inseminate thousands of females in any year.

For example, it may be desirable to bias the sex ratio in calf crops so as to produce more heifers in dairy herds. This enhances genetic selection pressure relating to production and confirmation of traits. It also allows for fewer calving difficulties, prevents the production of freemartins and bull calves, and enables longer lactation periods. Each of these results lead to more profits for the dairy herder.

A further basis on which to bias or skew the sex ratio within calf crops is to produce more male offspring in beef herds. This can enhance the quality and profitability of those herds or may be used to produce the next generation of herd bulls in intensively raised beef cattle.

In certain circumstances it is not possible to obtain sexed semen already packaged in straws. For example, sexed semen will not be available from quarantined, unproductive, or long dead sires. As such, it simply is presently not possible to obtain, in a convenient manner, sexed semen from such sires.

By way of a further example, in the swine industry a bias towards a near exclusive female piglet litter is advantageous because of the issue of boar taint, which is commonly associated with the presence of undesired chemicals present, not only in the gut (i.e. Skatole), but mostly in cells of the male testis (i.e. Androstenone) that develop during puberty. Boar taint can affect the flavour of pork meat to the extent that the meat is not acceptable for human consumption and, as such, a pig litter biased towards the female gender is advantageous.

Many approaches have been used previously to influence sex selection, including sperm being separated by their sex via flow cytometry for artificial insemination (AI). Semen collected for AI is introduced into a breeding female's uterus for the purpose of achieving a pregnancy through fertilisation. In certain circumstances it is not commercially viable to inseminate mammals to effect fertilisation. For example, multiparous mammals such as pigs require a larger volume of semen and costly antibodies in order to obtain a sizeable litter and, as such, there is a need to provide a simple and cost effective alternative to what has previously been used in the art. In addition, it may be commercially impractical, except in gilt AI, to supply sufficient sexed or unsexed male sperm in conjunction with antibodies from an external source to obtain a satisfactory bias towards a near exclusive female piglet litter.

The Applicant has previously demonstrated a method that was directed to the influencing of sex selection in artificial insemination, that method using the exposure of unsexed semen to antibodies to sex specific antigens present on spermatozoa carrying chromosomes of an undesired sex, typically the male sex, whereby the spermatozoa to which the sex specific antigens are bound are generally unable to effect fertilisation of an egg, such that fertilisation is likely to be effected by spermatozoa carrying chromosomes of a desired sex. The spermatozoa carrying chromosomes of the undesired male sex and to which the sex specific antigens are bound are unable to penetrate the Zona Pellucida surrounding the egg. This method required that the exposure of the unsexed semen to the sex specific antibodies occurred during the physical act of artificial insemination. Particularly, this exposure occurred as the unsexed semen was injected into a uterus of the subject of artificial insemination. This method is described, for example, in the Applicant's Australian Patent 2010324528.

Applicant's Australian Patent 2010324528 also describes a method for influencing sex selection in artificial insemination, the method characterised by the steps of:

Positioning a biasing plug means in relation to a semen containing straw, such that as the unsexed semen contained within the straw is ejected it must pass through the biasing plug means; and

As the unsexed semen passes through the biasing plug means it is forced into intimate contact with sex specific antibodies provided in the biasing plug means, those antibodies having been chosen to bind to sex specific antigens on spermatozoa carrying chromosomes of an undesired sex,

whereby the intimate contact of the semen and the antibodies either in the biasing plug means or thereafter causes binding therebetween, and by which the spermatozoa carrying chromosomes of the undesired sex and to which the sex specific antigens are bound are generally unable to effect fertilisation of an egg, thereby ensuring substantially that fertilisation is likely to be effected by spermatozoa carrying chromosomes of a desired sex. The sex specific antibodies from within the biasing plug means may also have been ejected therefrom into the uterus of the animal being inseminated along with the semen from the straw, the spermatozoa to which the sex specific antibodies are bound are then generally unable to effect fertilisation of an egg, thereby ensuring substantially that fertilisation may only be effected by spermatozoa carrying chromosomes of a desired sex.

The methods of the Applicant's Australian Patent 2010324528 described were found not to be as successful as anticipated as the sex specific antigens did not bind the ‘male’ spermatozoa efficiently in the lower reaches of the uterus of the animal being inseminated.

One object of the method of the present invention is to overcome substantially the above mentioned problems of the prior art, or to at least provide a useful alternative thereto.

Each document, reference, patent application or patent cited in this text is expressly incorporated herein in their entirety by reference, which means that it should be read and considered by the reader as part of this text. That the document, reference, patent application, or patent cited in this text is not repeated in this text is merely for reasons of conciseness.

Reference to cited material or information contained in the text should not be understood as a concession that the material or information was part of the common general knowledge or was known in Australia or any other country.

Throughout this specification, unless the context requires otherwise, the word “comprise”, or variations such as “comprises” or “comprising”, will be understood to imply the inclusion of a stated integer or group of integers but not the exclusion of any other integer or group of integers.

### SUMMARY OF THE INVENTION

In accordance with the present invention there is provided a method for biasing sex selection, the method comprising the introduction into a uterus of a subject a volume of micro-particle conjugates, the micro-particle conjugates being proportioned so as to approximate the size and shape of spermatozoa and thereby be carried by peristaltic waves through the uterus and fallopian tubes to the infundibulum, at which point any spermatozoa present or arriving thereafter undergo capacitation and expose antigens that may be bound by anti-male antibodies provided in the micro-particle conjugates, the binding of the exposed antigens on the spermatozoa by the anti-male antibodies resulting in their inability to penetrate the Zona Pellucida of an egg and effect fertilisation, the micro-particle conjugates comprising a micro-particle of a biodegradable substance, to which are bound anti-male sperm antibodies, and a sacrificial coating surrounding the micro-particle and bound anti-male sperm antibodies, wherein the sacrificial coating is removed by the same enzymes in the infundibulum that cause the capacitation of the spermatozoa therein.

Preferably, the spermatozoa may be introduced to the uterus of the subject by either natural reproductive or artificial means. Still preferably, the volume of micro-particle conjugates are introduced into the uterus of the subject either prior to or at the same time as the spermatozoa.

In one form of the present invention, the sacrificial coating of the micro-particle conjugates is formed of one or more lipids.

Preferably, the sacrificial coating of the micro-particle conjugates is formed of cholesterol.

In a further form of the present invention, the biodegradable substance forming the micro-particle is agarose.

In accordance with the present invention there is further provided a micro-particle conjugate comprising a micro-particle of a biodegradable substance, at least one anti-male sperm antibody, and a sacrificial coating, wherein the at least one anti-male sperm antibody is bound to an outer surface of the micro-particle and the sacrificial coating surrounds both the micro-particle and the or each antibody the sacrificial coating being formed such that it is subject to removal by enzymes present in the infundibulum of a subject that cause the capacitation of spermatozoa therein.

Preferably, the sacrificial coating of the micro-particle conjugates is formed of one or more lipids.

Still preferably, the sacrificial coating of the micro-particle conjugates is formed of cholesterol.

Preferably, the biodegradable substance forming the micro-particle is agarose.

In one form of the present invention a plurality of micro-particle conjugates are provided in a volume of diluent. Preferably, the resulting diluted suspension of micro-particle conjugates is provided in the form of straws, bottles or tubes.

In a further form of the present invention there is provided a mixture of a volume of micro-particle conjugates as described hereinabove, and unsexed spermatozoa, for administration to a subject.

Where the subject is bovine the mixture is preferably one part micro-particle conjugate and one part unsexed spermatozoa. Where the subject is porcine the mixture is preferably one part micro-particle conjugate and two parts unsexed spermatozoa.

In accordance with the present invention there is still further provided a bispecific monoclonal anti-male antibody.

Preferably, bispecific monoclonal anti-male antibody is suitable for use in the method and apparatus of the present invention.

Still preferably, the bispecific monoclonal anti-male antibody is an HY anti-male sperm antibody.

### BRIEF DESCRIPTION OF THE DRAWINGS

The present invention will now be described, by way of example only, with reference to one embodiment thereof and the following figures, in which:

FIG. 1 is a diagrammatic cross-sectional representation of an apparatus for biasing sex selection in accordance with the present invention, showing a micro-particle conjugate proportioned so as to approximate the size and shape of a spermatozoa;

FIG. 2 is a diagrammatic representation of the apparatus for biasing sex selection of

FIG. 2, shown with the sacrificial cholesterol coating removed and showing the anti-male sperm antibody bound to antigens on the surface of a capacitated sperm;

FIG. 3 is a diagrammatic representation of an ovary, a fallopian tube and one horn of a uterus, showing in particular the relationship of the ovary, and in turn any egg released thereby during ovulation, to the infundibulum of the fallopian tube;

FIG. 4 is a diagrammatic representation of the process of capacitation of a spermatozoa, shown in stages 1, 2 and 3 over which the cholesterol outer layer coating the spermatozoa is removed and the spermatozoa becomes capable of fertilising an egg, being FIGS. 4(a), 4(b) and 4(c), respectively;

FIG. 5 is a graphic representation of the 21 day bovine oestrus cycle, showing the period during which it is best to utilise/administer a pre-mating straw; and

FIG. 6 is a graphic representation of the hormone levels during the bovine oestrus cycle.

### DETAILED DESCRIPTION OF THE INVENTION

The present invention provides a method for biasing sex selection, the method comprising the introduction into a uterus of a subject a volume of micro-particle conjugates, wherein the micro-particle conjugates are proportioned so as to approximate the size and shape of spermatozoa and thereby be carried by peristaltic waves through the uterus and fallopian tubes to the infundibulum, at which point any spermatozoa present or arriving thereafter undergo capacitation and expose antigens that may be bound by anti-male antibodies provided in the micro-particle conjugates, the binding of the exposed antigens on the spermatozoa by the anti-male antibodies resulting in their inability to penetrate the Zona Pellucida of an egg and effect fertilisation.

The spermatozoa are introduced to the uterus of the subject by either natural reproductive or artificial means. The volume of micro-particle conjugates are introduced into the uterus of the subject either prior to or at the same time as the spermatozoa.

The micro-particle conjugates comprise a micro-particle of a biodegradable substance, for example agarose, to which are bound anti-male sperm antibodies. In addition, the micro-particle conjugates further comprise a sacrificial coating, for example a lipid or cholesterol coating, surrounding the micro-particle and anti-male sperm antibodies. The sacrificial coating is removed by the same enzymes in the infundibulum that cause the capacitation of the spermatozoa therein.

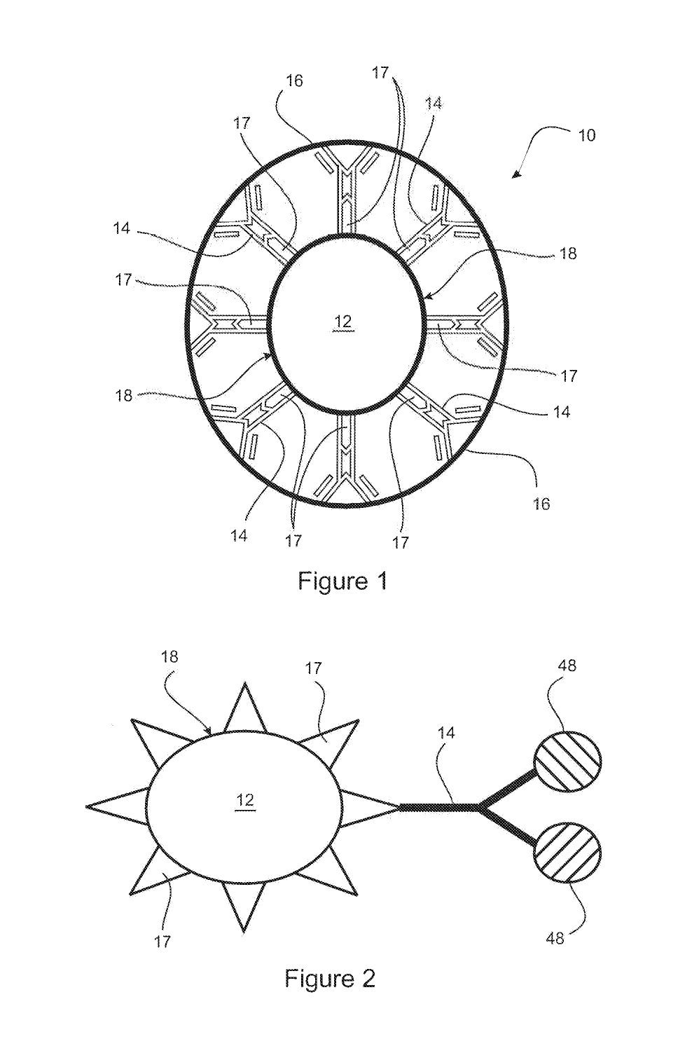

In FIG. 1 there is shown a micro-particle conjugate 10 comprising a micro-particle 12 of a biodegradable substance, at least one anti-male sperm antibody 14 bound thereto, and a sacrificial coating, for example a cholesterol coating 16. The at least one anti-male sperm antibody 14 is bound to a specific antigen 17 provided on an outer surface 18 of the micro-particle 12, and the cholesterol coating 16 surrounds both the micro-particle 12 and the or each antibody 14.

The biodegradable substance forming the micro-particle is, in one form of the present invention, agarose.

The anti-male sperm antibody 14 is in one form of the present invention a bispecific monoclonal anti-male sperm antibody. The bispecific monoclonal anti-male sperm antibodies are prepared from a suitable pair of hybridomas. However, it is understood by the Applicants that other anti-male sperm antibodies may be equally applicable to the method and apparatus of the present invention, including other monoclonal antibodies, and polyclonal and nano antibodies.

It is envisaged that a plurality of micro-particle conjugates are provided in a volume of diluent, for example either egg-yolk citrate buffer (equal volumes of 3.2 per cent sodium citrate, pH adjusted to 6.8 with citric acid, and egg-yolk) or egg-yolk phosphate buffer (equal volumes of phosphate buffer, pH 7.4 (2 per cent Na2HPO4. 12H2O and 0.2 per cent KH2PO4 and egg-yolk). These buffers may be used with or without combinations of sulphanilamide (1.5 mg/ml), penicillin (500 units/ml) and streptomycin (500 pgm/ml). The preparation of this combination of micro-particle conjugates and diluent is achieved using a milk homogenizer, the cholesterol coating of the conjugates acting to keep them in suspension.

The resulting diluted suspension of micro-particle conjugates may be provided or packaged in the form of straws, bottles or tubes.

It is further envisaged that the present invention provides in another form a mixture of a volume of micro-particle conjugates as described hereinabove, and unsexed spermatozoa, for administration to a subject.

Where the subject is bovine the mixture is, for example, one part micro-particle conjugate and one part unsexed spermatozoa. Where the subject is porcine the mixture is, again for example, one part micro-particle conjugate and two parts unsexed spermatozoa.

A consideration of the anatomy and physiology of the uterine or fallopian tubes, otherwise known as the oviducts, is useful in considering and understanding the present invention and its manner of operation.

In FIG. 2 there is shown the micro-particle conjugate 10 in which the sacrificial cholesterol coating 16 has been stripped away through capacitation, to be described hereinafter. One anti-male sperm antibody 14 is shown bound to two antigenic proteins of one or more capacitated spermatozoa, to also be described hereinafter.

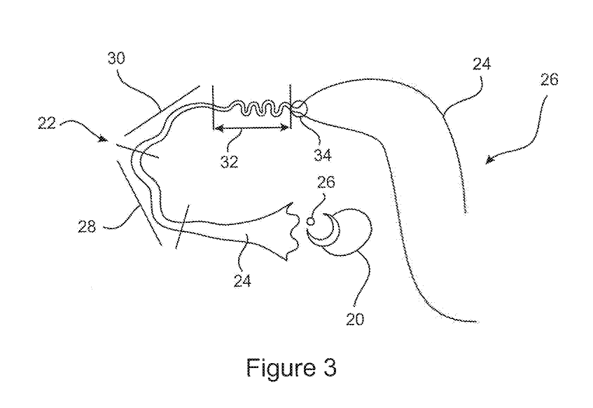

In FIG. 3 there is shown a diagrammatic representation of an ovary 20, a fallopian tube 22 and one horn 24 of a uterus 26 (shown in part only). Each fallopian tube 22 is about 10 to 13 cm long and 1 to 2.5 cm in cross-sectional diameter. The channel of the tube 22 is lined with a mucous membrane layer that has many folds and papillae. Over the mucous layer are 3 layers of muscle tissue. The innermost layer of muscle tissue has spiral fibres, the middle layer has circular fibres, and the outer sheath has longitudinal fibres that end in many finger-like branches (fimbriae) near the ovaries 20, forming a funnel-like depositary, the infundibulum 24.

The infundibulum 24 catches and channels any released egg, for example egg 26, and is the widest distal (uppermost) portion of each fallopian tube 22. The endings of the fimbriae extend over the ovary and they contract close to the ovary's surface during ovulation in order to guide the freed egg. Leading from the infundibulum 24 are the long central portions of the fallopian tube called the ampulla 28, the ampullary-isthmic junction 30, and the isthmus 32. The isthmus 32 then connects at the intramural duct 34, located in the top portion (fundus) of the uterus, to the one horn 24 of the uterus 26. The intramural duct 34 leads through the thick uterine wall to the uterine cavity, where fertilised eggs normally attach and develop. The channel of the intramural duct 34 is the narrowest part of the fallopian tube 22, but still easily capable of letting the micro-particle conjugates 10 pass through.

The micro-particle conjugates 10 are about 5×8 μm, and are minute relative to the lumen of the uterine tubes (>50 μm2). Peristaltic waves during the 4 day oestrous cycle phase comfortably squeeze the micro-particle conjugates 10 along the length of the tube 22 and up into the infundibulum 24 where they need to remain for a period of time to be most effective.

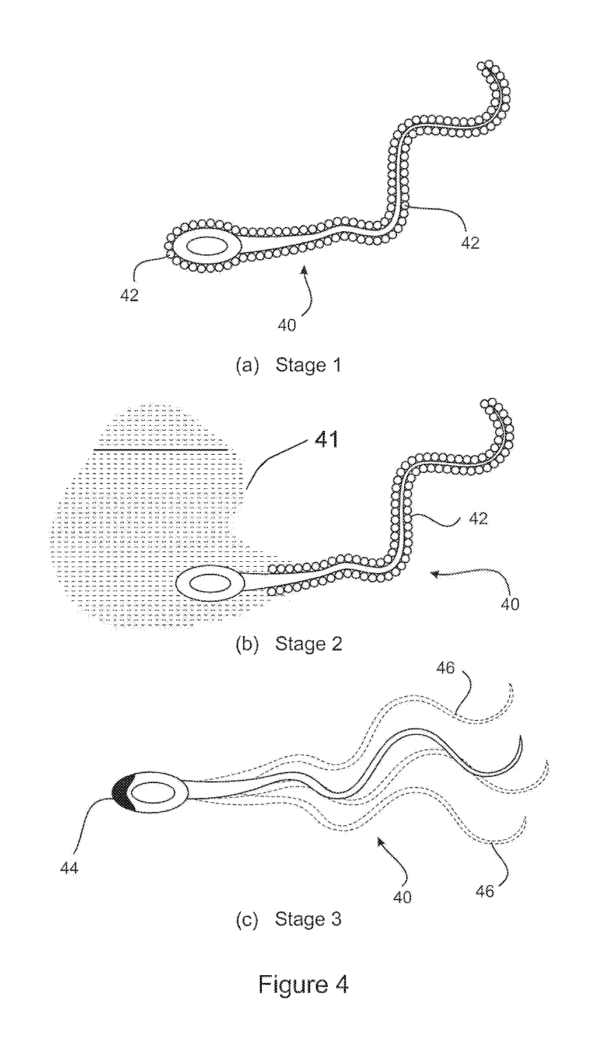

In FIGS. 4(a), (b) and (c) there is shown a diagrammatic representation of the process of capacitation of a spermatozoa 40, shown in stages 1 (ejaculated sperm), 2 (sperm within the infundibulum) and 3 (sperm near the Zona Pellucida), respectively, over which the cholesterol outer layer coating the spermatozoa is removed and the spermatozoa becomes capable of fertilising an egg.

Capacitation is the penultimate step during the maturation of mammalian sperm and is required to render them competent to fertilise an oocyte. This is a biochemical step: the spermatozoa move normally and look mature prior to capacitation. In vivo this step typically occurs after ejaculation within top end of the reproductive tract. In vitro, capacitation can occur by incubating spermatozoa that have either undergone ejaculation or have been extracted from the epididymis in a defined medium for several hours. The uterine mucus aids with the capacitation, secreting sterol-binding albumin, lipoproteins, as well as proteolytic and glycosidasic enzymes and heparin, shown generally as natural conception lubrication 41 in FIG. 4(b).

Capacitation is the functional maturation of the spermatozoon 40. The changes take place via the sperm cell membrane in which it is thought that antibody receptors are made available through the removal of a glycoprotein or cholesterol layer 42, as seen best with reference to FIGS. 4(b) and 3(c). The area of the acrosomal cap 44 is also altered such that the acrosome reaction becomes possible by exposing further proteins, as seen best in FIG. 4(c). It is also the step during which the tail becomes hyper activated, shown at 46.

Only the capacitated spermatozoa 40 have the antigenic proteins 48, shown in FIG. 2, exposed on their surface for the male sex specific antibodies 14 of the micro-particle conjugates 10 to interact with them (i.e. attach themselves). As described above, the micro-particle conjugates 10 comprise a bio-degradable agarose and are coated with a cholesterol layer 16. The latter is dissolved in the same manner and in the same place as experienced during the capacitation of sperm 40, by enzymes within the infundibulum, as depicted in FIGS. 3(a), 3(b) and 3(c).

The method and apparatus of the present invention may be better understood with reference to the following non-limiting example.

### EXAMPLE

Premating straws for bovine use contain a mixture of micro-particle conjugates 10 and a diluent, described hereinabove. Alternatively, for artificial insemination (AI), straws having one part micro-particle conjugates 10 and one part unsexed semen are provided. Premating flasks or bottles for porcine use also contain a mixture of micro-particle conjugates 10 and a diluent. Again, for artificial insemination (AI), tubes having one part micro-particle conjugates 10 and one part unsexed semen are provided.

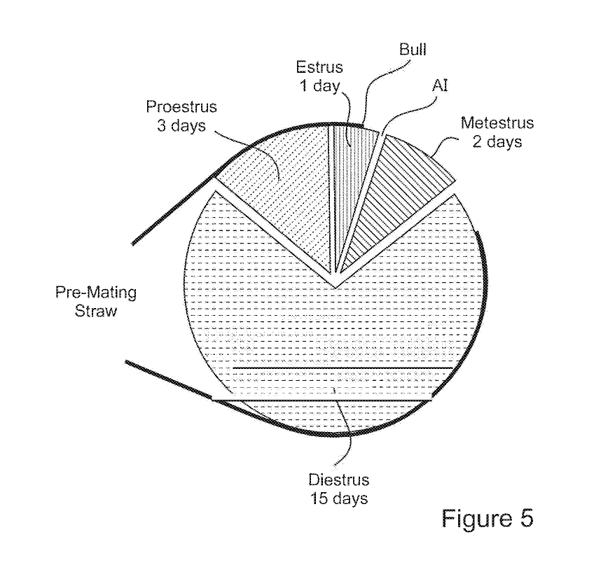

Once a cow is ready to be bred again for her next calf, inseminate her anytime with a pre-mating straw, preferably around 50 days post calving, but at the latest at the same time as the normal Al, as illustrated with reference to FIG. 5.

After heat detection, breed to a bull (natural reproduction) or use AI at the subsequent morning, respectively evening milking with a preferred, previously unsexed, semen straw.

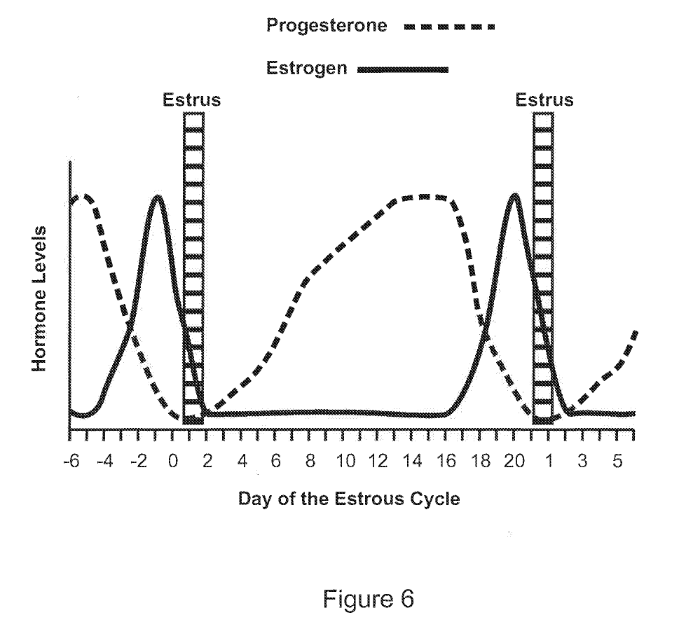

Uterine contractions (that is, the peristalsis which is synchronous with the estrous cycle) transport the content of the pre-mating straw (being micro-particle conjugates 10 and a diluent) towards the infundibulum into which the egg is released. The contractions begin prior to standing heat due to the rise in Estrogen levels in the developing follicle, as shown in FIG. 6, which illustrates cyclic changes in reproductive hormones.

As can be seen with reference to the above description, the method and apparatus of the present invention provide a greatly expanded and/or improved applicability of the immunogenic action of anti-male sperm antibodies, for example the bispecific monoclonal HY anti-male antibody described herein, by facilitating their positioning in the female uterine tract (i.e. in the infundibulum) to ensure they are located in the same place as capacitating sperm.

Capacitating sperm are stripped of their protective sugar and cholesterol coatings by enzymes and lipoproteins released by the mucus lining of the infundibulum, thereby laying bare antigens, such as antigens 48, which would not otherwise be accessible to an antibody reaction. The improvement stems from conjugating the anti-male antibodies 14 onto micro-particles 12 that resemble sperm heads, for example in shape and size. The uterus and associated tubes during oestrous experience peristaltic waves akin to swallowing that transport sperm and our conjugation constructs up towards the distal end of the fallopian tubes (i.e. the infundibulum that envelops at least partially the ovary during oestrus). If un-conjugated antibodies alone are inserted into the uterus then they will remain in the uterus due to them being too small to be affected by the uterine peristaltic waves.

As described above, coating the micro-particle conjugates with cholesterol mimics sperm, but it also aids in protecting them, as they might remain in the uterus for up to 18 days awaiting the next oestrus, from both natural phagocytic actions and also from agglutinating with each other. The sterol-binding albumin and lipoproteins, as well as proteolytic and glycosidasic enzymes and heparin reactions near the ovary will liberate the conjugated antibodies to perform their intended function, to bind to anti-male sperm antigens. The micro-particles simply dissolve.

Modifications and variations such as would be apparent to the skilled addressee are considered to fall within the scope of the present invention.

1-19. (canceled)

20. A method for biasing sex selection, the method comprising the introduction into a uterus of a subject a volume of micro-particle conjugates, wherein the micro-particle conjugates being proportioned so as to approximate the size and shape of spermatozoa and thereby be carried by peristaltic waves through the uterus and fallopian tubes to the infundibulum, at which point any spermatozoa present or arriving thereafter undergo capacitation and expose antigens that may be bound by anti-male antibodies provided in the micro-particle conjugates, the binding of the exposed antigens on the spermatozoa by the anti-male antibodies resulting in their inability to penetrate the Zona Pellucida of an egg and effect fertilisation, the micro-particle conjugates comprising a micro-particle of a biodegradable substance, to which are bound anti-male sperm antibodies, and comprise a sacrificial coating surrounding the micro-particle and bound anti-male sperm antibodies, wherein the sacrificial coating is removed by the same enzymes in the infundibulum that cause the capacitation of the spermatozoa therein.

21. The method of claim 1, wherein the spermatozoa are introduced to the uterus of the subject by either natural reproductive or artificial means.

22. The method of claim 1, wherein the volume of micro-particle conjugates are introduced into the uterus of the subject either prior to or at the same time as the spermatozoa.

23. The method of claim 1, wherein the sacrificial coating of the micro-particle conjugates is formed of one or more lipids.

24. The method of claim 1, wherein the sacrificial coating of the micro-particle conjugates is formed of cholesterol.

25. The method of claim 1, wherein the biodegradable substance forming the micro-particle is agarose.

26. The method of claim 1, wherein the anti-male antibodies are bispecific monoclonal anti-male antibodies, and optionally HY anti-male sperm antibodies.

27. A micro-particle conjugate comprising a micro-particle of a biodegradable substance, at least one anti-male sperm antibody, and a sacrificial coating, wherein the at least one anti-male sperm antibody is bound to an outer surface of the micro-particle and the sacrificial coating surrounds both the micro-particle and the or each antibody, the sacrificial coating being formed such that it is subject to removal by enzymes present in the infundibulum of a subject that cause the capacitation of spermatozoa therein.

28. The micro-particle conjugate of claim 27, wherein the sacrificial coating of the micro-particle conjugates is formed of one or more lipids.

29. The micro-particle conjugate of claim 27, wherein the sacrificial coating of the micro-particle conjugates is formed of cholesterol.

30. The micro-particle conjugate of any one of claim 27, wherein the biodegradable substance forming the micro-particle is agarose.

31. The micro-particle conjugate of claim 27, wherein a plurality of micro-particle conjugates are provided in a volume of diluent.

32. The micro-particle conjugate of claim 31, wherein the resulting diluted suspension of micro-particle conjugates is provided in the form of straws, bottles or tubes.

33. The micro-particle conjugate of claim 27, wherein the anti-male antibody is a bispecific monoclonal anti-male antibody, and optionally an HY anti-male sperm antibody.

34. A mixture of a volume of micro-particle conjugates according to 27, and unsexed spermatozoa, for administration to a subject.

35. The mixture of claim 33, wherein the subject is bovine and the mixture is one part micro-particle conjugate and one part unsexed spermatozoa.

36. The mixture of claim 33, wherein the subject is porcine and the mixture is one part micro-particle conjugate and two parts unsexed spermatozoa.
